(19) United States
(12) Patent Application Publication
(10) Pub. No.: US 2023/0346013 A1
(43) Pub. Date: Nov. 2, 2023

(54) Eco-Friendly Personal Exhalant Filter

(71) Applicant: Philter Labs Incorporated, Encinitas, CA (US)

(72) Inventors: John Grimm, Santee, CA (US); Andrew Lamoureux, Santee, CA (US); Gianni Grimm, Santee, CA (US)

(73) Assignee: Philter Labs Incorporated, Encinitas, CA (US)

(21) Appl. No.: 18/130,544

(22) Filed: Apr. 4, 2023

Related U.S. Application Data
(63) Continuation-in-part of application No. 17/885,432, filed on Aug. 10, 2022.
(63) Continuation-in-part of application No. 17/712,393, filed on Apr. 4, 2022.
(63) Continuation-in-part of application No. 17/087,792, filed on Nov. 3, 2020.
(63) Continuation-in-part of application No. 17/068,719, filed on Oct. 12, 2020.
(63) Continuation-in-part of application No. 16/998,459, filed on Aug. 20, 2020.
Application No. 17/068,719 is a continuation of application No. 16/820,553, filed on Mar. 16, 2020, now Pat. No. 10,834,963.
Application No. 16/820,553 is a continuation of application No. 16/257,881, filed on Jan. 25, 2019, now Pat. No. 10,588,344.
Application No. 16/257,881 is a continuation of application No. 15/884,082, filed on Jan. 30, 2018, now Pat. No. 10,470,498.
(60) Provisional application No. 63/327,271, filed on Apr. 4, 2022.
(60) Provisional application No. 63/233,175, filed on Aug. 13, 2021.
(60) Provisional application No. 63/170,543, filed on Apr. 4, 2021.
(60) Provisional application No. 62/931,312, filed on Nov. 6, 2019.
(60) Provisional application No. 62/889,814, filed on Aug. 21, 2019.
(60) Provisional application No. 62/452,871, filed on Jan. 31, 2017.

(51) Int. Cl. A24F 3/00; A24F 13/00; B01D 53/02; B01D 46/00; A24F 40/40; A24D 3/17; B01D 46/24; B01D 53/04; A24F 7/04; B01D 39/16; A61L 9/014; A24F 40/485

(52) U.S. Cl. CPC A24F 7/04; A24D 3/17; A24F 3/00; A24F 13/00; A24F 40/40; A24F 40/485; A61L 9/014; B01D 39/1692; B01D 46/003; B01D 46/0036; B01D 46/0038; B01D 46/2411; B01D 53/02; B01D 53/0407; A61L 2209/13; B01D 2239/045; B01D 2253/102; B01D 2257/80; B01D 2257/90; B01D 2259/4541

(57) ABSTRACT

As an accessory to cannabis and other smokers, an increased capacity, environmental and ecological friendly, sustainable solution personal air filter is disclosed. The solution effectively and efficiently combines passive filter medium and properties of fluid mechanics to achieve the increased capacity. Additionally, a specific order of passive filter elements is provided to increase filtering efficiency. Wherever possible, biodegradable materials are employed such a biodegradable PLA polymers.

### CROSS-REFERENCE TO RELATED APPLICATIONS

This patent application claims the benefit of priority date of U.S. Prov. Pat. App. Ser. No. 63/327,271 filed on Apr. 4, 2022 entitled “Eco-Friendly Personal Air Filter.” Additionally, the present patent application is a Continuation-in-Part of U.S. Pat. App. Ser. No. 17/712,393 entitled “Personal Air Filter Utilizing Mechanical, Chemical and Electrostatic Filter Media.” filed on Apr. 4, 2022 which claims benefit of the priority date of U.S. Prov. Pat. App. Ser. No. 63/370,543 filed on Apr. 4, 2021.

Still further, the present patent application is a Continuation-in-Part of U.S. Pat. App. Ser. No. 17/068,719 entitled “Low Emissions Electronic Smoking Device and Emissions Filtering Device, filed on Oct. 12, 2020 which is a Continuation of U.S. Pat. App. Ser. No. 16/820,553 filed on Mar. 16, 2020, now U.S. Pat. No. 10,834,963, which is a Continuation of U.S. Pat. App. Ser. No. 16/257,881, filed on Jan. 25, 2019, now U.S. Pat. No. 10,588,344, which is a Continuation-in-Part of U.S. Pat. App. Ser. No. 15/884,082, filed on Jan. 30, 2018, now U.S. Pat. No. 10,470,498 which claims benefit of the priority date of U.S. Prov. Pat. App. Ser. No. 62/452,871 filed on Jan. 31, 2017.

Yet further, the present patent application is a Continuation-in-Part of U.S. Pat. App. Ser. No. 17/885,432, entitled “Ionic Personal Exhalant Filter,” filed on Aug. 10, 2022 which claims benefit of the priority date of U.S. Prov. Pat. App. Ser. No. 63/233,175 filed on Aug. 13, 2021.

Still further, the present patent application is a Continuation-in-Part of U.S. Pat. App. Ser. No. 17/087,292, entitled “Electronic Cigarette Filter Assembly,” filed on Nov. 3, 2021 which claims benefit of the priority date of U.S. Prov. Pat. App. Ser. No. 62/931,312 filed on Nov. 6, 2019.

Yet still further, the present patent application is a Continuation-in-Part of U.S. Pat. App. Ser. No. 16/998,459, entitled “Smart Personal Air Filter,” filed on Aug. 20, 2020 which claims benefit of the priority date of U.S. Prov. Pat. App. Ser. No. 62/889,814 filed on Aug. 21, 2019.

Accordingly, the entire contents of all the aforementioned U.S. patent submissions are hereby expressly incorporated by reference.

### BACKGROUND OF THE INVENTION

### Field of the Invention

The present invention pertains generally to accessories for electronic smoking devices and to smoking in general. More specifically, the present invention relates to an increased capacity personal exhalant air filter capable of removing exhale pollutants and eliminating odors, also providing an ecological friendly solution.

### Description of the Prior Art

Ever increasing in popularity, “electronic cigarettes/e-cigarettes” and vaporizers typically use an electrical heating element to heat a liquid usually containing a mixture of nicotine and flavorings/extract (or cannabis including THC or CBD) that produces a mist similar to smoke but without the excessive heat that causes combustion.

Also, an emerging acceptance of cannabis user’s and legalization thereof has resulted in emerging market and public needs. Particularly users often desire to not offend others, for example in living spaces such as houses, apartments or hotels, with second hand smoke and any ramifications thereof. Or, users may simply wish to remain discrete. Still further, typical users wish to encourage environmental and ecological friendly options wherever possible.

Hence, it is an object of the present invention to provide an increased capacity person exhale filter device utilizing an effective combination of passive filter mediums and fluid mechanics designs. Still further, it is an object of the present invention to appeal to earth-minded consumers that desire sustainable solutions. Hence, therefore, it is an additional object of the present invention to provide a device comprising biodegradable, compostable, recyclable and/or reusable material wherever possible.

### BRIEF SUMMARY OF THE INVENTION

The present invention specifically addresses and alleviates the above-mentioned deficiencies, more specifically, the present invention, in a first aspect is a personal exhalant air filter, or air purifier, for filtering exhale. The filter may be characterized as comprising: a mouthpiece; an outer body; and an inner cartridge encased by the outer body and containing a filter medium (i.e., a plurality of passive filter elements), the inner cartridge containing the filter medium being biodegradable and is able to decay naturally without harming the environment in three years or less.

The invention in this aspect is additionally characterized in that it further comprises an end (i.e., exit vent) cap including apertures (or exit vent holes) allowing air discharge, the end cap further covers an opening at a distal end of the outer body, the opening providing access for removal and replacement of the inner cartridge. Herein the filter medium is also a composite filter medium with different types of passive filter medium in succession including: a copper (or metallic) mesh for filtering passive ion elements; a first carbon filter medium; a HEPA (high efficiency particulate air) filer medium, wherein the first carbon filter medium precedes the HEPA filter medium providing greater absorption to the carbon filter medium and providing less clogging to the HEPA filter medium. Additionally, the composite filter medium includes a second carbon filter medium and a nylon hydrophobic mesh.

The invention in this first aspect also includes a convergent-divergent nozzle downstream of the mouthpiece reducing back pressure and facilitating exhale.

In a second aspect, the invention is directed to an ecological friendly personal air purifier for filtering user’s exhale comprising: an outer body; a mouthpiece overmolded to and overlapping the outer body wherein the mouthpiece is injection molded to the outer body; a filter medium at an interior to the outer body, the filter medium further comprising a carbon filter medium; and an endcap having exit vent holes removably coupled to an end of the outer body opposite the mouthpiece. The carbon filter medium further comprises extruded carbon subsequently baked to a specific shape and having vertical apertures therethrough for a passage of exhale.

The invention in this second aspect is additionally characterized as further comprising an inner cartridge containing the filter medium, and further the inner cartridge made from biodegradable bioplastic. A filter medium includes: a first carbon filter; a HEPA (high efficiency particulate air) filter having pleated (or corrugated and juxtaposed) panels; and a second carbon filter wherein the first copper filter precedes the HEPA filter and the second carbon filter succeeds the HEPA filter.

A compostable sleeve surrounding the personal air filter is also included, the compostable sleeve further comprising seeds embedded therein. In various embodiments and invention aspects, the outer body further comprises a soft material covering an outer surface thereof, the soft material for providing and improving a look and feel to the personal air filter. The filter medium employed herein is more specifically a plurality of passive filter elements, wherein the inner cartridge containing the filter medium is biodegradable and is able to decay naturally without harming the environment in three years or less.

Importantly in this and other aspects, the end cap is snap fit to the distal end of the outer body; additionally, the end cap comprises a protrusion and the outer body has a corresponding aperture receiving said protrusion.

While the apparatus and method has or will be described for the sake of grammatical fluidity with functional explanations, it is to be expressly understood that the claims, unless expressly formulated under 35 USC §112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC §112 are to be accorded full statutory equivalents under 35 USC §112, or similar applicable law. The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an isometric view of a preferred embodiment, eco-friendly personal exhalant air filter, or air purifier of the present invention;

FIG. 2 is an exploded view showing preferred assembly;

FIG. 3 is an additional exploded view illustrating replaceable inner cartridge mechanical filter medium;

FIG. 4 is an elevational view of a preferred inner cartridge;

FIG. 5 is a top plan view of the inner cartridge particularly showing snap-in top tray having condensation ports arranged in a 3 ✕ 3 matrix;

FIG. 6 is cross-sectional view of the inner cartridge taken along line 6-6, in FIG. 2 illustrating condensation ports;

FIG. 7 is an enlarged view of multiple convergent-divergent nozzles employed by the present invention taken along line 7-7 in FIG. 6;

FIG. 8 is an additional perspective view of the personal air filter from an underneath vantage point; and

FIG. 9 is a prospective view illustrating an invention feature including a compostable sleeve containing the personal air filter.

### DETAILED DESCRIPTION OF PREFERRED EMBODIMENTS

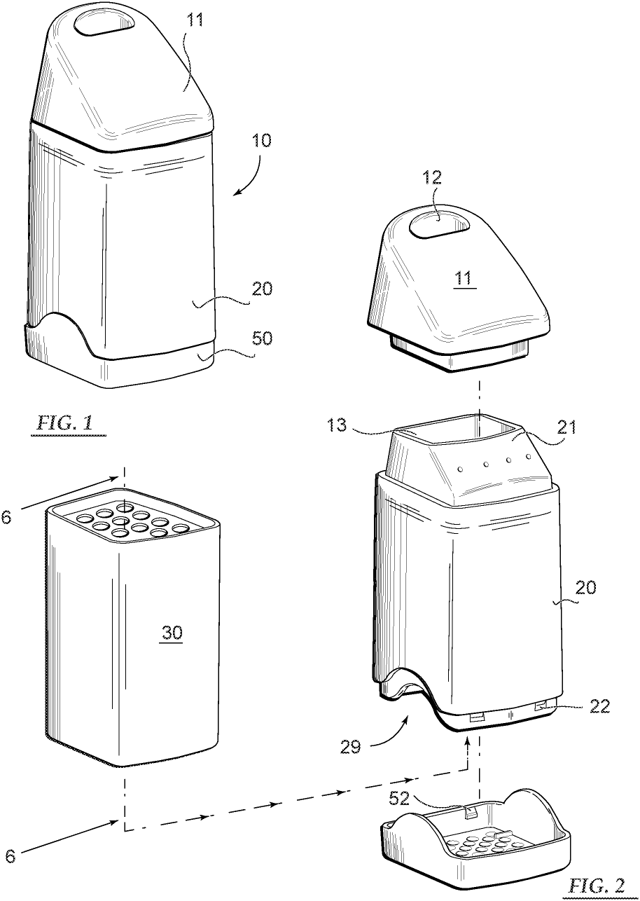

Referring initially to FIG. 1 and FIG. 2, isometric and exploded views are provided illustrating connections and molding features of a personal exhalant air filter, or alternatively person air purifier 10. Generally, a main body outer enclosure, or outer body 20 is flanked by a top cover mouthpiece 11 and a bottom end cap 50, also referred to as exit vent herein. The connections are either overmolded friction fit or snap-in as detailed herein. More particularly, the outer body 20 has an upper portion 21 extending at its proximal. Initially, the outer body 20 is provided to a separate mold. Next, the mouthpiece 11 is formed by injection molding over the upper portion 21 forming a tight seal. At the opposite end, the end cap is snap fit to the outer body. Particularly, the end cap 50 has protrusions 52 to mate to corresponding apertures 22 extending from the outer body 20.

Also with regard to FIG. 1 and FIG. 2, the plastic main body outer enclosure (i.e. outer body 20) is rigid; but however, it has been coated with soft material to improve feel and appearance. FIG. 2 illustrates an inner cartridge 30 that fits inside the outer body 20. Importantly, the inner cartridge 30 comprises 100% bio-sourced plastic made from renewal resources, e.g. PLA bioplastic, also known as polylactic acid made from, for example, plant starch such as corn or sugarcane. It is biodegradable in that is capable of being broken down naturally via microorganisms.

Still further with reference to FIG. 2, the upper portion 21 of the outer body 20, together with the mouthpiece 11, provide a larger air void 13 employed to promote convection and reduce back pressure. Hence, a user exhale into the device 10 via the mouthpiece 11 can be made more easily and naturally. Additionally, the inner cartridge 30 is replaceable; hence, the outer body 20 distal end has an opening 29 to remove and replace the inner cartridge 30 by removing and replacing end cap 50.

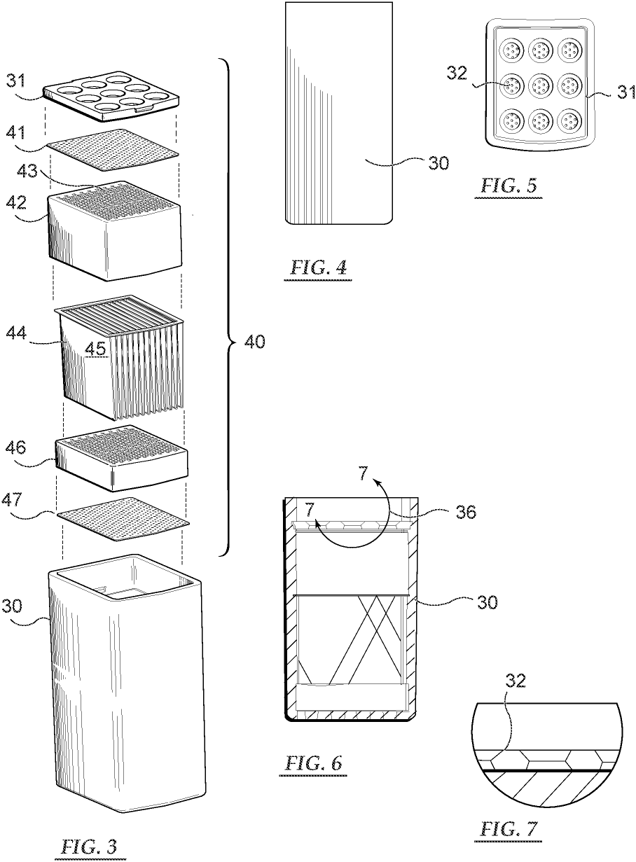

With reference to FIG. 3, an exploded view of the biodegradable inner cartridge 30 is shown particularly illustrating the passive filter medium 40. More specifically, the filter medium 40 is a composite filter medium in that several individual filter mediums 41, 42, 44, 46, and 47 are employed in sequence. The filter mediums 41, 42, 44, 46, 47 are being referred to as passive in that they are mechanical in nature, they do not require a power supply and they can function without an extra fluid prime mover, such as a fan, other than the fluid pressure provided by the exhale itself.

Further with regard to FIG. 3 and the filter mediums 40 provided in sequence, a metallic (e.g. copper) mesh 41 is provided attracting and capturing any ionic particles. Next, a carbon, or graphite filter medium 42 is provided. Specifically, the carbon is extruded to an exact shape, baked in for example an oven, and in doing so, further providing a plurality of apertures 43 therethrough for the passage of air.

Subsequent to the passive ion element copper mesh 41 and the first carbon filter 42, a pleated HEPA (high efficiency particulate air) filter medium 44 is provided. In a preferred embodiment, the HEPA filter has pleats or panels 45 that are folded. Alternatively, the panels 45 can be corrugated such that the panels 45 are wavy. Importantly, the order of the carbon filter medium 42 and the HEPA filter medium 44 is chosen carefully to allow for greater absorption into the carbon 42 and less clogging of the HEPA 44.

Yet still further with regard to FIG. 3, a second carbon filter medium 46 is provided further improving overall filter 40 efficiency. Last in the sequence, a nylon hydrophobic mesh 47 is provided tending to repel moisture. All mediums 49 are housed in a replaceable, biodegradable inner cartridge 30.

As stated with regard to FIG. 4, the biodegradable PLA inner enclosure 30 is more particularly made from 100% bio-sourced plastic made from renewal resources, for example, corn or sugar cane. Hence, the inner cartridge 30 is made of plant-based polymer making the personal air filter herein eco-friendly. Additionally with regard to FIG. 9, the eco-friendly filter 10 includes recyclable packaging and a feature that includes an outer sleeve 60 made from compostable paper with wildflower, or other seeds 61, embedded therein.

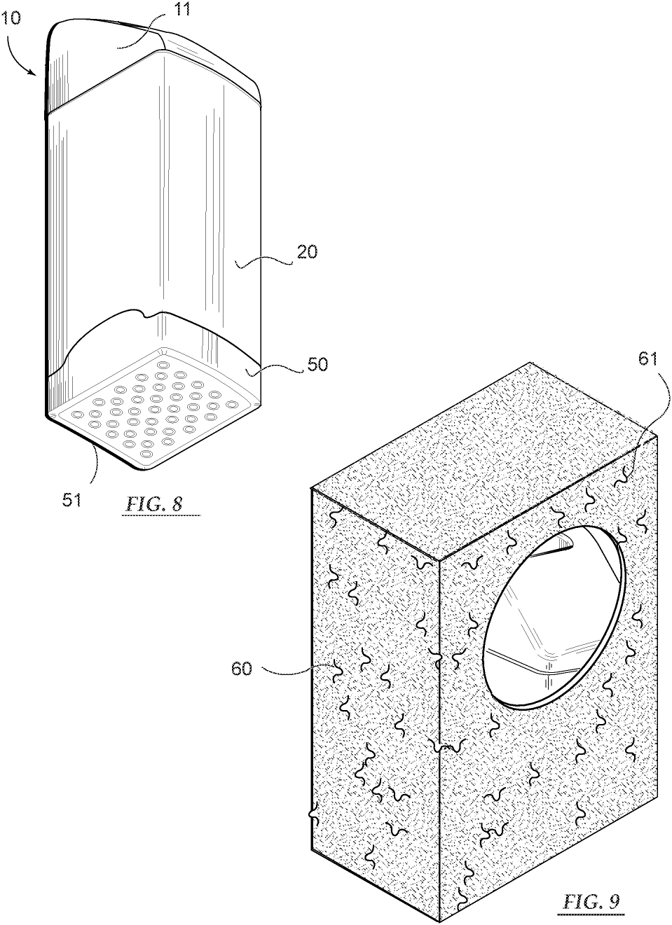

With reference to FIG. 5 a top view of the snap-in top tray 31 is provided. As shown, condensation ports are arranged in a 3 ✕ 3 matrix. FIG. 6 is a cross sectional view of an inner cartridge taken along line 6-6 in FIG. 2; while FIG. 7 provides an enlarged view of a snap in tray taken about line 7-7 in FIG. 6. It should be appreciated that the nine condensation ports are convergent-divergent nozzles 32 or de Laval nozzles. Lastly, FIG. 8 provides a view of the personal air purifier 10 from an underneath vantage point illustrating end cap 50 exit vent holes 51.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed above even when not initially claimed in such combinations.

While the particular Eco-Friendly Personal Exhalant Filter as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

What is claimed is:

1. An ecological friendly personal air purifier for filtering user’s exhale comprising:
a mouthpiece;
an outer body; and
an inner cartridge encased by the outer body and containing a filter medium, the inner cartridge containing the filter medium being biodegradable and is able to decay naturally without harming the environment in three years or less.

2. The ecological friendly personal air purifier for filtering user’s exhale of claim 1, further comprising an end cap including apertures allowing air discharge, the end cap further covering an opening at a distal end of the outer body, the opening providing access for removal and replacement of the inner cartridge.

3. The ecological friendly personal air purifier for filtering user’s exhale of claim 1, wherein the filter medium is a composite filter medium, the composite filter medium comprising:
a carbon filter medium; and
a HEPA (high efficiency particulate air) filer medium, wherein the carbon filter medium precedes the HEPA filter medium allowing exhaled air to pass through the carbon filter medium first, and subsequently the HEPA filter medium, providing greater absorption to the carbon filter medium and providing less clogging to the HEPA filter medium.

4. The ecological friendly personal air purifier for filtering user’s exhale of claim 1, further comprising a convergent-divergent nozzle downstream of the mouthpiece.

5. An ecological friendly personal air purifier for filtering user’s exhale comprising:
an outer body;
a mouthpiece overmolded to and overlapping the outer body wherein the mouthpiece is injection molded to the outer body;
a filter medium at an interior to the outer body, the filter medium further comprising a carbon filter medium; and
an endcap having exit vent holes removably coupled to an end of the outer body opposite the mouthpiece.

6. The ecological friendly personal air purifier for filtering user’s exhale of claim 5, the carbon filter medium further comprising extruded carbon subsequently baked to a specific shape and having vertical apertures therethrough for a passage of exhale.

7. The ecological friendly personal air purifier for filtering user’s exhale of claim 5, further comprising an inner cartridge containing the filter medium the inner cartridge comprising biodegradable bioplastic.

8. The ecological friendly personal air purifier for filtering user’s exhale of claim 5, wherein the outer body comprises the filter medium via an inner cartridge, the inner cartridge comprising a convergent-divergent nozzle.

9. The ecological friendly personal air purifier for filtering user’s exhale of claim 5, the filter medium further comprising:
a copper mesh filter medium; and
a nylon hydrophobic mesh.

10. The ecological friendly personal air purifier for filtering user’s exhale of claim 5 wherein the carbon filter medium is a first carbon filter medium, the filter medium further comprising:
a HEPA (high efficiency particulate air) filter having pleated panels; and
a second carbon filter wherein the first copper filter precedes the HEPA filter and the second carbon filter succeeds the HEPA filter.

11. The ecological friendly personal air purifier for filtering user’s exhale of claim 5, further comprising a compostable sleeve surrounding the personal air filter, the compostable sleeve further comprising seeds embedded therein.

12. The ecological friendly personal air purifier for filtering user’s exhale of claim 5, the outer body further comprising soft material covering an outer surface thereof, the soft material for providing and improving a look and feel to the personal air filter.

13. The ecological friendly personal air purifier for filtering user’s exhale of claim 5, wherein the end cap is snap fit to the end of the outer body, the end cap comprising a protrusion and the outer body comprising a corresponding aperture receiving said protrusion.

14. An ecological friendly personal air purifier for filtering user’s exhale comprising:
a mouthpiece;
an outer body; and
a filter medium for filtering user’s exhale; and
a convergent-divergent nozzle downstream of the mouthpiece and upstream of the filter medium.

15. The ecological friendly personal air purifier for filtering user’s exhale of claim 14, further comprising an inner cartridge containing the filter medium the inner cartridge comprising biodegradable bioplastic.

16. The ecological friendly personal air purifier for filtering user’s exhale of claim 14, the filter medium further comprising an inner cartridge containing a HEPA (high efficiency particulate air) filter comprising a plurality of folded pleats.

17. The ecological friendly personal air purifier for filtering user’s exhale of claim 14, further comprising a convergent-divergent nozzle downstream of the mouthpiece.
